(19) United States
(12) Patent Application Publication
(10) Pub. No.: US 2009/0298165 A1
(43) Pub. Date: Dec. 3, 2009

(54) Process for removing selenium from air or water

(76) Inventors: Jay M. Wiedemann, Powellton, WV (US); Richard Huffman Squire, South Charleston, WV (US); Richard C. Schoening, Charleston, WV (US)

Correspondence Address: WEST VIRGINIA UNIVERSITY RESEARCH CORPORATION, 886 CHESTNUT RIDGE ROAD, P.O. BOX 6224, MORGANTOWN, WV, 26506-6224, US

(21) Appl. No.: 12/381,911

(22) Filed: Mar. 18, 2009

Related U.S. Application Data
(60) Provisional application No. 61/070,209, filed on Mar. 20, 2008.

(51) Int. Cl. C12M 1/12

(52) U.S. Cl. 435/297.1

(57) ABSTRACT

A process for removing selenium from air or water can use proteins naturally found in bacteria able to live in selenium rich environments to capture and/or reduce the selenium to an insoluble form that can be captured.

### CROSS-REFERENCE TO RELATED APPLICATIONS

This application claims priority from application No. 61/070,209 filed on Mar. 20, 2008.

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

### BRIEF DESCRIPTION OF THE SEVERAL VIEWS OF THE DRAWING

FIG. 1 is a continuous selenium removal process.

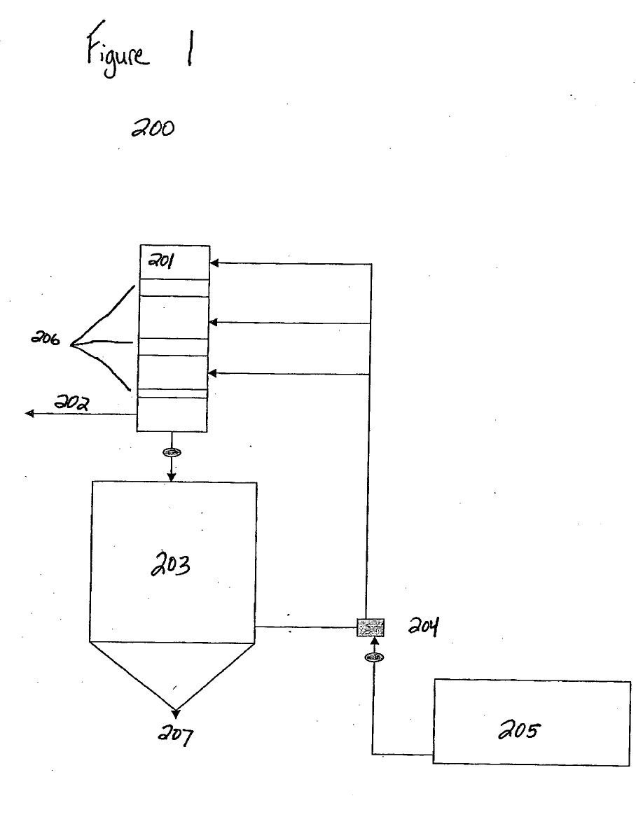

### DETAILED DESCRIPTION OF THE INVENTION

Naturally occurring bacteria are able to eliminate and to break down selenium in the environment. Eight enzyme systems have been identified that are involved in an overall process to reduce selenium to a harmless state naturally. The Sel proteins within the natural systems are selenium reductase, iron selenide clusters, selenocysteine lyase, nitrate reductase, nitrite reductase, selenophosphate synthesase, S-methyltransferase, glutathione reductase, and superoxide dismutase. The Sel proteins can be any of the recognized amino acid sequences for each of the different Sel proteins and the conservative variants thereof. Conservative variants are amino acid sequences that may differ from the normal sequence; however, the tertiary protein still has virtually the same binding features as the normal protein sequence. Conservative variants are often a replacement of one or more amino acids with those of similar properties on the side chain. A Sel complex, Sel enzyme complex, or Sel protein complex, as used in this application, is one or more Sel proteins capable of the capture and reduction of selenium when utilized in concert such as by competent bacteria.

Sel compotent bacteria are able to reduce selenium the ions selenide⁽⁻²⁾, selenite⁽⁺⁴⁾, and selenate⁽⁺⁶⁾ to insoluble selenium. The proteins selenate reductase, selenite reductase, and selenocysteine lyase have primary selenium binding functions. The other three Sel proteins have non-selenium primary functions; however, since selenium has the right characteristics for these proteins that have a lack of fidelity, they also process selenium.

A Sel protein, Sel enzyme complex or Sel protein complex can be found in many bacterial species and can also be transformed and expressed in E. coli or any other competent bacteria. Some bacteria found naturally having Sel proteins expressed are Bacillus megaterium, Bacillus cereus, Clostridium butyricum, Staphylococcus aureus pI258, Streptomyces lividans, Streptomyces pRJ28, Exiguobacterium sp., Pseudomonas sp. ED-23, Pseudomonas stuizeri OX pPB, Serratia marcescens DUI358, Pseudomonas aeruginosa Tn501, Alcaligenes pMER610, Shigella flexneri Tn21, Pseudomonas sp. ADP, Xanthomonas campestris Tn5044, Xanthomonas sp. Tn5053, Pseudomonas fluorescens, Shewanella putrefaciens pMERPH, Thiobacillus ferrooxidans, Pseudoalteromonas haloplanktis, Acidithiobacillus ferrooxidans SUG 2-2, Acidithiobacillus ferrooxidans MON-1. Expressed proteins can be isolated from the bacteria by standard biochemical techniques such as affinity chromatography, other chromatography techniques, and other standard techniques within microbiology. The reduction of selenium can be performed by one or more of the Sel competent bacteria in a standard column. An example would be one or more Sel competent colonies suspended on a support in a column with selenium being passed through the column. While amounts of selenium and passage rates may vary, an example would be about 400 ppb of an oxidized form of selenium being passed through a column at about 20 ml/hour with selenium being non-detectable at the column exit (less than 1 ppb). At much higher concentrations (64,000 ppb selenate), about 30% of the selenate can be reduced to elemental selenium as evidenced by the elemental selenium red precipitate. Sel competent bacteria will show no signs of toxicity at these levels of selenate. In another embodiment the process would take place in an anaerobic environment.

After the Sel protein has been expressed and isolated, or alternatively using the native Sel protein complex, the Sel protein or protein complex can be used as a chelating agent to remove selenium from any selenium containing environment. A Sel protein may be used in a solution to bind selenium where any of the Sel proteins may be reversibly attached to a removal apparatus. The reaction of the Sel proteins with selenium to form a chelate (chemical complex) in the lab has been established using actual industrial coal samples and other industrial solutions/slurries. In a selenium chelating process the removed selenium can be collected at a high concentration for conversion to Se⁽⁰⁾ by the Sel protein complex. With the appropriate design either a Sel Protein or the Sel protein complex can be used to remove dilute selenium from water or air. For low levels of selenium in water or air, a column with active protein or protein complex can be suitable as any properly designed column configuration known to one skilled within the art can provide suitable contact time.

A typical example of a process to remove selenium from an aqueous environment is the attachment of the Sel protein or the Sel protein complex to a removal apparatus designed to provide the necessary residence time to react with the volume of effluent desired from a selenium containing source. A removal apparatus can be beads, a support column, a column packed with a Sel protein or the Sel protein complex, fiber mats or any other means for attaching the Sel protein or Sel protein complex. There are numerous attachment possibilities for either the Sel protein or Sel complex known to one skilled in the art such as protein attachment to a removal apparatus by any conventional ligand attachment method such as a Strep-Tag from Sigma-Genosys. The proper operation is to use standard methods so that the protein or protein complexes do not pack too tightly so as to allow uniform diffusion through the bed or column. One or more removal apparatuses can be added directly to the air or water sample possibly containing selenium. Further, one or more removal apparatuses can be held within a housing apparatus that contains an inlet and outlet for desired water or air flow through the housing apparatus. A housing apparatus can be of any design such as partially or totally enclosed and the removal apparatus can be held by any standard means including either permanently or removeably held within the housing apparatus. The housing can also contain one or more low pressure devices. Redistributors are standard in this technology as well as angled bed supports with multiple diffusion supports that maintain a low pressure drop through the beds. Back flushing is also a common practice to insure proper distribution. The volume of the effluent may be increased or decreased by changing the cross section and/or volume of the removal apparatus for use in different sized ponds. Existing technology such as an in-line selenium monitor to detect selenium in solution can be used to detect when the binding of the Sel protein and selenium in the removal apparatus nears reaction completion. When a first removal apparatus is nearly completely reacted, the selenium containing effluent can be switched to a second removal apparatus while the first is regenerated. After the first apparatus is removed for regeneration the second apparatus is brought on-line by a means of exchange which can be any means standard to one skilled within the art. The regeneration may be performed by a means to regenerate. The means to regenerate can be one of two methods: either a very specific concentration of a salt such as guanidine hydrochloride, or raising the protein to an elevated temperature between about 55° C. to about 65° C. The salt method of regeneration can be performed by using an amount of guanidine hydrochloride that can vary from about 0.35M to about 2.90M to denature the Sel protein in steps. The steps denature a portion of the protein at a time. The regeneration step performs two functions. The first function is the release of the selenium from the Sel protein or Sel complex attached to the removal apparatus where the selenium is released into a concentrated solution. The concentrated solution can then be reduced from Se⁽⁺⁶⁻⁺²⁾ to Se⁽⁰⁾ by a means of reduction such as Sel complex capable bacteria or chemical treatment to form an insoluble compound. The means of reduction will produce Se⁽⁰⁾ which is then collected by either phase separation or centrifugation, depending on the concentration. The second function of regeneration is to allow the Sel protein to be renatured quickly either by dropping the temperature or the salt concentration slowly with appropriate buffering so the column can be brought back on-line in an expeditious manner. The only known impediment to the above process is a very high concentration of selenate, -ite, etc, in the inlet stream (>150,000 ppb). In this case special modifications such as intermediate solids (elemental selenium) collection to prevent elemental selenium from blocking the column or reactor need to be in place as pluggage could occur. One means to modify can be accomplished by using stirred tank reactors in series, with an intermediate hold tank large enough to allow the elemental selenium to settle to the bottom of the hold tank. The remaining material from the top of the intermediate tank is processed further. In cases where very large amounts of selenium are present in the various oxidation states several stirred tank reactors and hold tanks can be placed in series for selenium reduction with no plugging.

A potentially more cost-effective alternative to current selenium removal techniques is a continuous bioreactor 200 packed with living colonies of mixed native and/or transformed bacteria capable of producing a Sel protein complex. The bioreactor may additionally include enhanced bacteria. The selenium removal activity of the native bacteria is enhanced through culturing in sequentially increasing levels of selenium. The enhancement of the bacteria can be achieved by a progressive increasing of Se content in a growth media during successive generations of bacterial growth. A growth media can be any conventional bacterial growth media such as one containing phenol red mannitol, litmus milk, tryptic soy broth, urea, nutrient broth and LB broth, a combination of tryptone, yeast extract, and sodium chloride, pH 7.0 with no selenium. Sel competent bacteria can then be transferred into a growth media with varying levels of Se added such as three different medias with 0.001, 0.005, and 0.01 nM Se⁽⁺⁶⁻⁺²⁾ respectively. The bacteria are then incubated 24 hours and checked for bacteria growth. The colony with both the highest Se concentration and bacterial growth can be used as a baseline. From that baseline fresh growth media with the baseline level of Se can be inoculated every 24 hours for a week to create stock bacteria able to grow at the baseline. Using the same procedure as above, the selenium concentration can then be raised to successively higher levels: 0.003 mM, 0.01 mM, 0.05 mM, 0.1 mM, 0.2 mM, 0.4 mM, 0.6 mM, and finally 1 mM Se⁽⁺⁶⁻⁺²⁾ while adding in stock samples and freezing samples for recovery, if needed. Following the freezing practice, enhanced bacteria can be kept indefinitely and used at will. The method of enhancement would also work for the increased ability of bacteria to eliminate any other materials the bacteria has the ability to eliminate in normal conditions.

The bioreactor could be comprised of a filter with the filter which is a housing containing an inlet 201 and an outlet 202 for effluent to enter and exit, Sel protein complex competent bacteria, and a means to adhere the bacteria within the filter. The means of attachment for the bacteria within the filter could be beds such as cellulose or fiber mats or any other means known to one skilled in the art. The velocity of the effluent may be increased or decreased by changing the cross section and/or volume of the filter. The living bacteria in the filter could receive nutrients such as Luria-Bertani medium from a nutrient tank 203 wherein the nutrients can be added to the filter through a means to transfer nutrients such as a pump 204 or other conventional means known to one skilled in the art. In addition, the bioreactor can include a bacterial supply 205 for the ability to re-supply the bioreactor with Sel competent bacteria. The bacterial supply can be a holding tank or any other supply mechanism known to one skilled in the art. The bacterial supply has additional Sel complex competent bacteria growing within for addition to the bioreactor. The bacterial supply can also have a means to transfer the bacteria from the bacterial supply to the bioreactor. The means to transfer can be a pump or any other means of transfer known to one skilled in the art. The bioreactor may also include one or more protein supports 206 which can be made of any material known to one skilled in the art such as fiber or cellulose mats, wood chips, peat moss, or any other standard material. The bioreactor can also provide a controlled environment for the bacteria to optimize the selenium reduction. Conditions such as temperature and pH can be monitored by a means to monitor conditions such as thermocouples placed appropriately in the bed to insure temperature gradients are noted. The means to adjust the temperature, especially in winter can be a heat exchanger in which the warm, selenium free exit water heats the incoming effluent-laden stream. Additional heat input can be provided by standard heating techniques such as electric or natural gas heaters or bottled propane heaters. The pH can also be monitored by a standard pH meter for aqueous solutions and adjusted by standard caustic/acid additions common to those skilled in the art of wastewater treatment. However, in a large coal impoundment pond the pH is not expected to vary much. In an industrial stream some pretreatment of the effluent to be treated may be necessary using a means of pretreatment standard in the chemical industry. A bioreactor could be used with liquid or gas streams containing dilute amounts of selenium with the emphasis on streams that have contacted coal or byproducts from coal burning power plants. The bioreactor may also have a means for the collection of selenium 207 such as phase separation and centrifugation. Alternatively, a standard chemical waste water treatment system containing only the Sel protein complex can be used for removing selenium from large volumes of contaminated water.

Each of the above examples could be installed in the byproduct exit stream of either a bag house or an electrostatic precipitator or a flue gas desulfurizer scrubber unit. With some stream modification to insure the protein or living bio colony is not destroyed, the process could also be effectively used to remove selenium from effluent streams so these streams could be used for the manufacture of gypsum or concrete products without selenium present. In addition, the processes could be further modified so that they may be installed on the flue gas stream after various units (ESP, FGD, etc) have performed their tasks and preferably just prior to the exit stack. A modest amount of dilution air for temperature control and/or other oxidizing agents such as aluminum foil can be added to insure Se⁽⁰⁾ is fully oxidized. The Se⁽⁰⁾ can then be oxidized to Se⁽⁺²⁾ by an oxidizer such as a dilute permanganate solution scrubber or a column packed with aluminum wool or thin aluminum strips to increase the metal surface area and the selenium can be quantitatively captured by the Sel complex and/or Sel bioreactor colony. Aluminum acts as a solid state battery for the oxidation of Se. A metal to metal electron transfer at ambient temperatures begins almost instantaneously.

In a further embodiment a combination zero valent iron (ZVI) process is combined with the use of Sel competent bacteria. In ZVI the rate of selenate removal is reduced relative to other contaminants available in the sample. The use of a combination process with Sel competent bacteria could prevent the selenium breakthrough in the even of incomplete conversion of selenium that could be caused by the increase in the concentration of the other competing ions in the feed or poor water flow and/or distribution due to insoluble salts that precipitate out of solution. The addition of water to the ZVI process would also prevent insoluble salt or elemental selenium from dropping out of solution.

In a further embodiment the Sel competent bacteria can be used in combination with a Zhang-Frankenberger Rice Straw process that process selenium contaminated water through a series of at least three bioreactor channels filled with rice straw plants. The addition of Sel competent bacteria would reduce the selenium in the exit water of the bioreactor.

These terms and specifications, including the examples, serve to describe the invention by example and not to limit the invention. It is expected that others will perceive differences, which, while differing from the forgoing, do not depart from the scope of the invention herein described and claimed. In particular, any of the function elements described herein may be replaced by any other known element having an equivalent function.

What is claimed is:

1. A selenium removal device comprising one or more Sel proteins attached to one or more removal apparatuses.

2. The selenium removal device of claim 1 further comprising a housing apparatus to hold one or more removal apparatuses said housing apparatus further comprising an inlet and an outlet.

3. The selenium removal device of claim 2 further comprising a low pressure device wherein said low pressure device is one or more of a redistributor, angled bed support, and diffusion support.

4. The selenium removal device of claim 2 further comprising a back flushing mechanism.

5. The selenium removal device of claim 1 wherein said Sel proteins are one or more of selenate reductase, selenite reductase, and selenocysteine lyase.

6. The selenium removal device of claim 1 wherein said removal apparatus is one or more of beads, support columns, columns packed with Sel protein, or fiber mats.

7. The selenium removal device of claim 2 further comprising an intermediate solid collection.

8. The selenium removal device of claim 7 further comprising stirred tank reactors in a series between said inlet and said outlet.

9. The selenium removal device of claim 2 wherein said removal apparatus is monitored for binding completion and a 1^{st} removal apparatus is replaced in the housing apparatus with a 2^{nd} removal apparatus at a desired completion point.

10. The selenium removal device of claim 9 wherein said 1^{st} removal apparatus is regenerated.

11. A continuous bioreactor comprising a filter wherein said filter is a housing comprised of one or more bacteria capable of producing one or more Sel complexes capable of reducing the Sel ions selenide, selenite, and selenate to insoluble selenium, an inlet, an outlet, and a means to adhere said bacteria within said filter.

12. The continuous bioreactor of claim 11 wherein said bacteria are enhanced be adding Sel complex competent bacteria to a plurality of growth media wherein said growth media have varying levels of Se⁽⁺⁶⁻⁺²⁾, incubating said bacteria, creating a baseline from bacterial colonies growing within the highest level of Se within said plurality of growth media, using said baseline to grow a stock of said Sel competent bacteria then added said stock bacteria to successively higher Se⁽⁺⁶⁻⁺²⁾ levels until said bacterial colonies are able to grow in 1 mM Se⁽⁺⁶⁻⁺²⁾ growth media.

13. The continuous bioreactor of claim 11 wherein said means of attachment is a bed.

14. The continuous bioreactor of claim 11 further comprising one or more protein supports.

15. The continuous bioreactor of claim 11 further comprising a nutrient tank and a means to transfer nutrients from said nutrient tank to said filter.

16. The continuous bioreactor of claim 11 further comprising a bacteria supply reservoir and a means to transfer bacteria from said bacteria supply reservoir to said filter.

17. The continuous bioreactor of claim 11 further comprising a means to collect insoluble selenium.

18. The continuous bioreactor of claim 11 further comprising one or more of a means to monitor the conditions of said filter, a means to adjust the pH of said filter, a means to adjust the temperature of said filter, and a means of pretreatment before the selenium containing effluent is added to said bioreactor.

19. The continuous bioreactor of claim 11 further comprising the addition of a zero valent iron processes to said filter.

20. The continuous bioreactor of claim 11 further comprising the addition of a Zhang-Frankenberger Rice Straw process to said filter.
